SEQUENCE LISTING (16 sequences)

SEQ ID NO: 1 - protein, 278 aa, Arabidopsis thaliana
MNIVSWKDANDEVAGGATTRREREVKEDQEETEVRATSGKTVIKKQPTSISSSSSSWMKSKDPRIVRVSRAFGGKDRHSKVCTLRGLRDRRVRLSVPTAIQLYDLQERLGVDQPSKAVDWLLDAAKEEIDELPPLPISPENFSIFNHHQSFLNLGQRPGQDPTQLGFKINGCVQKSTTTSREENDREKGENDVVYTNNHHVGSYGTYHNLEHHHHHHQHLSLQADYHSHQLHSLVPFPSQILVCPMTTSPTTTTIQSLFPSSSSAGSGTMETLDPRQM

SEQ ID NO: 2 - DNA, 837 bp, Arabidopsis thaliana
atgaatatcgtctcttggaaagatgcaaacgacgaagttgcaggcggcgctacgacaagacgtgaaagagaagtaaaagaggatcaagaagaaaccgaagtcagagccaccagtggcaaaaccgtaattaaaaagcagcctacatcgatctcttcttcttcttcttcgtggatgaaatccaaggatccgaggattgttagggtttcacgcgcctttggaggcaaagaccgtcacagcaaagtgtgtacgttacgtggactacgtgacagacgcgtgagattatcagtcccaacggctattcagctctacgatcttcaagaacggctcggtgttgaccagcctagcaaagccgttgactggttgcttgatgcagctaaagaggagatcgacgagctacctccgttacctatctcgccggaaaatttcagcatcttcaaccatcatcagtccttcttgaatcttggtcaacggcccggtcaagatccgacccaactcgggtttaaaatcaatggatgtgtacaaaagtctactactactagccgcgaagaaaacgatagagagaaaggagaaaacgatgtcgtttacacaaacaatcatcatgttgggtcttatggaacttatcacaacctggaacatcatcatcatcatcaccaacatttgagtttacaggcagattatcatagtcatcaactacatagtcttgtcccatttccatcacaaattttggtatgtccaatgacgacatcaccaacaactacaactatacaatctttgtttccatcatcatcgtcagctggttcagggactatggagacattagatccgaggcaaatgtag

SEQ ID NO: 3 - protein, 10 aa, Arabidopsis thaliana
LDLELRLGFA

SEQ ID NO: 4 - DNA, 36 bp, Arabidopsis thaliana
gatctggatctagaactccgtttgggtttcgcttaa

SEQ ID NO: 5 - DNA, 2261 bp, Arabidopsis thaliana
acttggcaagattatgttctggtcacctgcataacaaaacaaaaacaatgtaatccaagcaaacaggtaacataacaatatttgattaataaacaaactaaataaacaattaccagaatgaggttcccagtgcttagcaatgaaattcttgaaatggatactagcagattggcgtacagaaagatcagagccaccatcaacaattatttgcaatatccttatcaaatgctgtggagtgtgctgcaactgaaaaaaaaaaccgaaatttcaatcagattttaaaccttaatagcagataatacaaccacagtcaaatcaagtcaatctaatgaagaaaaaaaacatcgatactccgaatttggataagcttagttctatcacaattctaaaataataaagagagaaattgaactaagcagagaggaaggaataaaaaccagaaaaagaaaaatttggtgtatacctgattaaggctctgctcagcagctctgcgctcgtcaggattgggactgaaagcagccgcgccaacgattaaagcgaggctaggcagatccatcggggaattcacaatcggaaagtcacgaatttcaagtctcctcccggaggaaatcgacgagactcgagagagtcaaatcaggaggaagaagagacgacgaagacgaaagagagagagaggtataagagaagaaggtctagggttttaaatggttcgttggtgtggttaagtctctctctctctctctcaaacgcgcgtggtaataagtgaaaaataaaataaaaaccagcacgtgacacacacaatacaaaaaccgagactttgcctggagctagtagtccttcacgccttactagtttacacggtcgatggacgcttttgttttttttggtccgtaaataattgggtttattatgtagactattaaacaaatattataataacaggacaaaatttcaaaattattcaataaatgaattgattgaattttttcactattttttaatttcttatttgattctttaatgattatattcaaaatagtaatatcctaacttcatatttaaactccattatgatcatggataggaatgaatttgtgaaaagtacataaaataattaaattagaaatatcctaaagaaccaaatgatattaatcataacaaaacaatacttctatacatgcaaatactctaattggagtatgttgtattaaaattttaatatggtagggaacactatttataaaagatattggagaacaaaaactttttataataattagcttagaaaaaaaatgatgagtaatcttctgaatttagtgggccaatccgacctaatagacaactcattgatttcatcttacgtcaggtacagttcatcaaatgcgaattatttcttaaattttaaaataatagctacttctttggttggttgcaaatgggcttcaggagtagcgcgggataaaatgggcctgtagaacatttgatgggccacaattctcaaaagcttttcttcctctttttttcttttgttcactgctctcaaaagtcaaaagctttccttttggtctcaaaagctcattatcattagggtagaaaaaagagtacatataagtggtaagttgtagtactagtaacatattaaatattaattctgtgaaaagttctttgatcacagtctttttttaataatcattacttgataaattatctacgtggaaaacaatacatcttacccccatttcatttggaggatgtacaatgtgtcaatgtgagactagccgttggatgggtctactcacattcgttgaagaacattaggtactgttaaaacttacaacctttttctcacccaatgtgcatttgcatgtgacatgcctagtgttactctttatctttttacacacataagtttcagtgcagatcagatgtttctctttttacattttgtcaacaagtttcacttttagctatcgtttacaaaagaaaaagtttcaaatatacaagaaaaatattaatcggcagtaaaatctctaagtccatttacggtaaccagagaatggttgttacactatgatcatcaagattaagaccaaacagtcacaagagatcttaaccattgattcaattttggtcaacgactcgaactcccaacataaccaaaacagtcagtcgtaataaaaagacgccccggtaggcgtataacagaacaacaacaccaaaagaaaagggaaaaaaaaaacagaggaactaataaacggacc

SEQ ID NO: 6 - DNA, 865 bp, Cauliflower mosaic virus
gcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgggggactctaga

SEQ ID NO: 7 - DNA, 250 bp, Arabidopsis thaliana
atatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 8 - DNA, 265 bp, Agrobacterium tumefaciens
gaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgg

SEQ ID NO: 9 - DNA, 75 bp, Artificial Sequence
  Description of Artificial Sequence Synthetic oligonucleotide
gatccacaattaccaacaacaacaaacaacaaacaacattacaattacagatcccgggggtaccgtcgacgagct

SEQ ID NO: 10 - DNA, 67 bp, Artificial Sequence
  Description of Artificial Sequence Synthetic oligonucleotide
cgtcgacggtacccccgggatctgtaattgtaatgttgtttgttgtttgttgttgttggtaattgtg

SEQ ID NO: 11 - DNA, 43 bp, Artificial Sequence
  Description of Artificial Sequence Synthetic oligonucleotide
gggctcgatctggatctagaactccgtttgggtttcgcttaag

SEQ ID NO: 12 - DNA, 43 bp, Artificial Sequence
  Description of Artificial Sequence Synthetic oligonucleotide
cttaagcgaaacccaaacggagttctagatccagatcgagccc

SEQ ID NO: 13 - DNA, 25 bp, Artificial Sequence
  Description of Artificial Sequence Synthetic oligonucleotide
atgaatatcgtctcttggaaagatg

SEQ ID NO: 14 - DNA, 27 bp, Artificial Sequence
  Description of Artificial Sequence Synthetic oligonucleotide
ctaccatgtaacaacgaatataatgtc

SEQ ID NO: 15 - DNA, 25 bp, Artificial Sequence
  Description of Artificial Sequence Synthetic oligonucleotide
cttggcaagattatgttctggtcac

SEQ ID NO: 16 - DNA, 41 bp, Artificial Sequence
  Description of Artificial Sequence Synthetic oligonucleotide
ggtccgtttattagttcctctgttttttttttcccttttct

(19) United States
(12) Patent Application Publication
(10) Pub. No.: US 2024/0140998 A1
(43) Pub. Date: May 2, 2024

(54) METHOD FOR INDUCING EMBRYOGENESIS OF SEED PLANT WITHOUT FERTILIZATION, PROTEIN USED THEREIN, NUCLEIC ACID, VECTOR, AND RECOMBINANT SEED PLANT IN WHICH EMBRYO CAN BE GENERATED WITHOUT FERTILIZATION

(71) Applicant: NATIONAL INSTITUTE OF ADVANCED INDUSTRIAL SCIENCE AND TECHNOLOGY, CHIYODA-KU, TOKYO (JP)

(72) Inventors: MASARU TAKAGI, Ibaraki (JP); MIHO IKEDA, Ibaraki (JP); NOBUTAKA MITSUDA, Ibaraki (JP); HIRONORI TAKASAKI, Ibaraki (JP)

(73) Assignee: NATIONAL INSTITUTE OF ADVANCED INDUSTRIAL SCIENCE AND TECHNOLOGY, CHIYODA-KU, TOKYO (JP)

(21) Appl. No.: 18/548,542

(22) PCT Filed: Mar. 2, 2022
(86) PCT No.: PCT/JP22/08956

§ 371 Date: Aug. 31, 2023

(30) Foreign Application Priority Data
Mar. 2, 2021 (JP) 2021-032750

(51) Int. Cl. C07K 14/415; C12N 15/82

(52) U.S. Cl. CPC C07K 14/415; C12N 15/8287; C07K 2319/00

(57) ABSTRACT

Provided is a method for artificially and efficiently inducing ectopic embryogenesis without fertilization in a seed plant. The present method includes introducing and expressing, in a seed plant, nucleic acid that includes a base sequence that codes a protein having an embryogenesis induction function.

### TECHNICAL FIELD

The present invention relates to a method for inducing embryogenesis in a seed plant without fertilization, a nucleic acid and a vector used for the method, and a recombinant seed plant produced by the method that can generate embryos without fertilization.

### BACKGROUND ART

Seeds of seed plants are mainly composed of embryos and endosperms. The embryos are infant plant individuals formed by development of fertilized eggs. The embryos germinate and then grow to be next-generation plants. The endosperms, which are tissues adjacent to the embryos, supply nutrients necessary for the growth of embryos for germination. The structure of each endosperm is divided into an inner endosperm derived from the embryo sac, which is a female gametophyte, and a perisperm derived from a sporophyte tissue. The embryos and endosperms are encapsulated in integuments to form ovules, which mature into seeds. The ovules are exposed in gymnosperms, whereas the ovules are covered with ovaries and the ovaries mature to form fruits in angiosperms.

In the angiosperm, the embryo sac contained in a pistil has an egg apparatus consisting of three cells including three antipodal cells at one end and a single egg cell at the other end. The embryo sac also has two polar nuclei at the center. When the pistil is pollinated, two sperm cells are carried into the embryo sac through a pollen tube extending from pollen, and the two sperm cells are fertilized with the egg cell and two polar nuclei in the embryo sac, respectively (this is called double fertilization). Fertilization of the egg cell (this is called reproductive fertilization) by one of the sperm cells produces a fertilized egg with a nuclear phase of 2n, which divides to form an embryo. Fertilization of two polar nuclei (this is called nutrient fertilization) by the other sperm cell produces an endosperm nucleus with a nuclear phase of 3n, which divides and proliferates to form an endosperm including an inner endosperm in the embryo sac.

In the angiosperm, an embryo usually does not develop unless fertilization (trophic fertilization) between a sperm cell and two polar nuclei occurs. However, it is known that an embryo may be produced without fertilization through a phenomenon called apomixis (also called asexual reproduction), in which seeds are produced by asexual reproduction without fertilization.

There are three known modes in which apomixis produces fertilization-free embryos in nature. The first mode is “diplospory,” which is found in, e.g., Guinea grasses. According to this mode, a diploid egg cell (2n) arises parthenogenetically from an embryo sac mother cell without meiosis, whereby the embryo is formed monogenetically. The second mode is “apospory,” which is found in, e.g., dandelions. According to this mode, a diploid embryo sac (2n) is formed not from the mother cell of an embryo sac but from a somatic cell in the vicinity thereof (nucellus), whereby the embryo is formed monogenetically. The third mode is “ectopic adventitious embryogenesis” (somatic embryonv or adventive embryony), which is widely seen in citrus and other plants. According to this mode, a nucellar cell in the ovule directly develops an adventive embryo (nucellar embryo) without forming an embryo sac.

Of these modes, the third, ectopic adventitious embryogenesis, has received particular attention from the standpoint of improving crop productivity. Specifically, ectopic adventitious embryogenesis usually produces, in addition to the fertilized embryo, multiple nucellar embryos, which are cloned embryos having the same genome as the parent plant. Therefore, if such ectopic adventitious embryogenesis can be artificially induced, it will be possible to produce a large number of clonal crops from multiple clonal embryos, which is extremely desirable from the perspective of improving production efficiency. For this reason, attempts have been made to artificially induce ectopic adventitious embryogenesis.

As examples of attempts to artificially induce such ectopic adventitious embryogenesis, there are reports using the gene LEC1 (LEAFY COTYLEDON1), a kind of transcriptional regulator in Arabidopsis species (Non-Patent Literature 1), and the gene BBM (BABY BOOM), which belongs to the AP2/ERF transcription factor family in Brassica species (Non-Patent Literatures 2 to 5). However, the formation rates of adventitious embryos using these genes were extremely low, about 13.6% in the case using the gene LEC1 and about 6.5% in the case using the gene BBM, both of which were far from practical levels.

### CITATION LIST

### Non-Patent Literature

[Non-Patent Literature 1] Lotan et al., Cell, 1998, Vol. 93, pp. 1195-1205
[Non-Patent Literature 2] Boutilier et al., Plant Cell, 2002, Vol. 14, pp. 1737-1749
[Non-Patent Literature 3] Srinivasan et al., Planta, 2007, Vol. 225, pp. 341-351
[Non-Patent Literature 4] Ouakfaoui et al., Plant Mol. Biol., 2010, Vol. 74, pp. 313-326
[Non-Patent Literature 5] Khanday et al., Nature, 2018, Vol. 565, pp. 91-95

### SUMMARY OF INVENTION

### Problems to be Solved

The problem addressed by the present invention is to provide a means to induce ectopic adventitious embryogenesis in a seed plant artificially and efficiently without fertilization.

### Means for Solving Problems

As a result of diligent investigation, the present inventors have found that a fusion protein of TCP13, a transcription factor derived from Arabidopsis thaliana, with SRDX, a transcription suppression element, has a function to induce ectopic adventitious embryogenesis with high efficiency when expressed in the cells of a seed plant, thereby arriving at the present invention.

The present invention relates to the following Aspects:
[Aspect 1] A method for inducing embryogenesis in a seed plant without fertilization, comprising expressing a protein having the function to induce embryogenesis in the seed plant,
   wherein the protein having the function to induce embryogenesis is a fusion protein comprising a first domain and a second domain fused together,
   wherein the first domain has an amino acid sequence with a sequence homology of 90% or more with the amino acid sequence of SEQ ID NO: 1, and
   wherein the second domain has an amino acid sequence with a sequence homology of 90% or more with the amino acid sequence of SEQ ID NO:3.
[Aspect 2] The method according to Aspect 1, wherein the expression of the protein having the function to induce embryogenesis is carried out by introducing a nucleic acid having a nucleotide sequence encoding the protein having the function to induce embryogenesis into the seed plant and expressing the protein having the function to induce embryogenesis in the seed plant,
   wherein the nucleic acid comprises a first coding region and a second coding region, wherein the first coding region encodes the first domain and the second coding region encodes the second domain, and
   wherein the nucleotide sequence of the first coding region and the nucleotide sequence of the second coding region are linked in-frame to each other.
[Aspect 3] The method according to Aspect 2, wherein the nucleic acid further comprises a promoter region, and wherein the nucleotide sequences of the first coding region and the second coding region are operably linked to the nucleotide sequence of the promoter region.
[Aspect 4] The method according to Aspect 2, wherein the nucleic acid is introduced into the genome of the seed plant in such a manner that the nucleotide sequences of the first coding region and the second coding region are operably linked to a sequence of an endogenous promoter region of the seed plant.
[Aspect 5] The method according to Aspect 3 or 4, wherein the protein having the function to induce embryogenesis is configured to be specifically expressed in an ovule under the control of the promoter region.
[Aspect 6] The method according to any one of Aspects 2 to 5, wherein the nucleic acid further comprises a terminator region, and wherein the nucleotide sequences of the first coding region and the second coding region are operably linked to the nucleotide sequence of the terminator region.
[Aspect 7] A protein having the function to induce embryogenesis in a seed plant without fertilization, comprising a fusion protein of a first domain and a second domain fused together,
   wherein the first domain has an amino acid sequence with a sequence homology of 90% or more with the amino acid sequence of SEQ ID NO:1, and
   wherein the second domain has an amino acid sequence with a sequence homology of 90% or more with the amino acid sequence of SEQ ID NO:3.
[Aspect 8] A nucleic acid encoding the protein according to Aspect 7, comprising a first coding region, which encodes the first domain, and a second coding region, which encodes the second domain, wherein the nucleotide sequence of the first coding region and the nucleotide sequence of the second coding region are linked in-frame to each other.
[Aspect 9] The nucleic acid according to Aspect 8, further comprising a promoter region, wherein the nucleotide sequences of the first coding region and the second coding region are operably linked to the nucleotide sequence of the promoter region.
[Aspect 10] The nucleic acid according to Aspect 9, which is configured to be introduced into the genome of the seed plant in such a manner that the nucleotide sequences of the first coding region and the second coding region are operably linked to a sequence of an endogenous promoter region of the seed plant.
[Aspect 11] The nucleic acid according to Aspect 9 or 10, which is configured to specifically express the protein having the function to induce embryogenesis in an ovule under the control of the promoter region.
[Aspect 12] The nucleic acid according to any one of Aspects 8 to 11, further comprising a terminator region, wherein the nucleotide sequences of the first coding region and the second coding region are operably linked to the nucleotide sequence of the terminator region.
[Aspect 13] A vector carrying the nucleic acid according to any one of Aspects 8 to 12.
[Aspect 14] The vector according to Aspect 13, which is a plant virus vector or an Agrobacterium vector.
[Aspect 15] A recombinant seed plant capable of generating embryos without fertilization, comprising the nucleic acid according to any one of Aspects 8 to 12 or the vector according to Aspect 13 or 14 incorporated into its cells.
[Aspect 16] A recombinant seed plant produced by the method according to any one of Aspects 1 to 6.

### Advantage of Invention

The present invention makes it possible to induce ectopic adventitious embryogenesis artificially and efficiently in a seed plant without fertilization.

### BRIEF DESCRIPTION OF DRAWINGS

FIG. 1 is a diagram illustrating the structure of Chimeric gene A (35S Pro:TCP13-SRDX_HSP ter) produced in Example 1.

FIG. 2 (a) is a photograph showing wild-type Arabidopsis thaliana (Col-0) at 21 days after seeding, and FIGS. 2 (b) to (h) are photographs each showing Arabidopsis thaliana Transformant plant A (T1) transformed with Construct A, which carries Chimeric gene A: (b) an aggregate of adventitious embryo-like structures at 21 days after seeding; (c) abnormal true leaves at 21 days after seeding; (d) a callus at 21 days after seeding; (e) aggregates of shoot-like structures at 268 days after seeding; (f) lines with variations in morphology at 192 days after seeding: (g) shoots at 226 days after seeding; and (h) flower organs at 57 days after seeding. The length of the black line segment in each photo corresponds to 5 mm in (a), 1 mm in each of (b), (c), (d), and (h), and in each of 1 cm in (e) and (f), respectively.

FIG. 3 is a graph indicating expression analysis results of differentiation-associated spermatogonial genes by qRT-PCR in Transformant plant A (T1) of Arabidopsis thaliana (Col-0) transformed with Construct A. These values are shown as relative values when the expression level of wild-type seedlings at 6 days after germination is set to 1, and corrected for the expression level of the gene UBQl as an internal standard.

FIG. 4 is a diagram illustrating the structure of Chimeric gene B (TT12 Pro:TCP13-SRDX_HSP ter) produced in Example 2.

FIG. 5 (a) is a confocal laser micrograph showing a transparency-treated seed obtained from wild-type Arabidopsis thaliana (Col-0) with transparency treatment, and FIGS. 5 (b) and (c) are confocal laser micrographs each showing a transparency-treated seed obtained from Arabidopsis thaliana Transformant plant B (T1), which was transformed with Construct B, which carries Chimeric gene B. Each of the seeds underwent a transparency treatment carried out using chloral hydrate in a conventional manner. (b) Two torpedo-shaped embryos are observed inside the single seed. (c) A heart-shaped embryo and a spherical embryo-like structure are observed inside the single seed. The length of the black line segment in each photo corresponds to 500 μm in each photograph.

### DESCRIPTION OF EMBODIMENTS

The present invention will now be described in detail with reference to specific embodiments. However, the present invention is not limited to the following embodiments and can be implemented in any form without departing from the spirit of the present invention.

The patent publications, published unexamined patent applications, and non-patent literature s cited in this specification are incorporated herein by reference in their entirety for all purposes.

### (I. Overview)

An aspect of the present invention provides a method for inducing embryogenesis in a seed plant without fertilization (hereinafter also referred to as “the embryogenesis-inducing method of the present invention,” or simply as “the method of the present invention”). According to an aspect of the present invention, the embryogenesis-inducing method of the present invention includes making a protein having the function to induce embryogenesis (hereinafter also referred to as “the embryogenesis-inducing protein of the present invention”) express in a seed plant. According to an aspect of the present invention, expression of the embryogenesis-inducing protein of the present invention is achieved by introducing a nucleic acid containing a nucleotide sequence encoding the embryogenesis-inducing protein of the present invention (hereinafter also referred to as “the embryogenesis-inducing nucleic acid of the present invention”) into the seed plant and have it expressed. According to an aspect of the present invention, introduction of the embryogenesis-inducing nucleic acid of the present invention into the seed plant is achieved by using a vector carrying the embryogenesis-inducing nucleic acid of the present invention (hereinafter also referred to as “the embryogenesis-inducing vector of the present invention”). According to an aspect of the present invention, the embryogenesis-inducing method of the present invention provides a recombinant seed plant that can cause embryogenesis without fertilization (hereinafter also referred to as “the recombinant seed plant of the present invention”).

In the following description, the embryogenesis-inducing protein of the present invention will be explained first, followed by the explanation of the embryogenesis-inducing nucleic acid of the present invention encoding the embryogenesis-inducing protein, and the embryogenesis-inducing vector carrying the embryogenesis-inducing nucleic acid. The subsequent explanation will be given of the embryogenesis-inducing method of the present invention employing the embryogenesis-inducing protein and/or the embryogenesis-inducing nucleic acid of the present invention, and finally, he recombinant seed plant of the present invention obtained by the embryogenesis-inducing method of the present invention will be explained.

### (2. Embryogenesis-Inducing Protein)

According to an aspect of the present invention, the embryogenesis-inducing protein of the present invention is a fusion protein containing a first domain and a second domain fused together.

The first domain of the embryogenesis-inducing protein of the present invention may have any amino acid sequence without restriction, but may preferably have an amino acid sequence identical or similar to the following amino acid sequence.

*The amino acid sequence of Arabidopsis thaliana transcription factor TCP13 (also referred to as “CR 117” or “RSE1”) protein (SEQ ID NO:1)

The second domain of the embryogenesis-inducing protein of the present invention may have any amino acid sequence without restriction, but may preferably have an amino acid sequence identical or similar to the following amino acid sequence.

*The amino acid sequence of SRDX protein, which is derived from a transcription suppression region of the Arabidopsis thaliana SUPERMAN gene with modifications (SEQ ID NO:3)

As is evident from the Examples described below, the fusion protein of the TCP13 protein and the SRDX protein has the effect of inducing embryogenesis when expressed in plant cells, and therefore can preferably be used as the embryogenesis-inducing protein of the present invention.

Proteins with similar amino acid sequences to this fusion protein can also preferably be used as the embryogenesis-inducing proteins of the present invention, since they are likely to have similar effects when expressed in plant cells due to their structural similarity.

Specifically, in the present invention, the amino acid sequence of the first domain of embryogenesis-inducing protein may preferably have a sequence homology (preferably, a sequence identity) of, e.g., 90% or more, or 95% or more, or 96% or more, or 97% or more, or 98% or more, or 99% or more, with the amino acid sequence of SEQ ID NO:1.

Likewise, in the present invention, the amino acid sequence of the second domain of embryogenesis-inducing protein may preferably have a sequence homology (preferably, a sequence identity) of, e.g., 90% or more, or 95% or more, or 96% or more, or 97% or more, or 98% or more, or 99% or more, with the amino acid sequence of SEQ ID NO:3.

The term “homology” between two amino acid sequences indicates the appearance rate of identical or similar amino acid residues at the respective positions when the two amino acid sequences are aligned, and the term “identity” between the two amino acid sequences indicates the appearance rate of identical amino acid residues at the respective positions when the two amino acid sequences are aligned.

In addition, “homology” and “identity” between two amino acid sequences can be determined by, for example, the Basic Local Alignment Search Tool (BLAST) program (Altschul et al., J. Mol. Biol., (1990), 215(3): 403-10).

### (3. Embryogenesis-Inducing Nucleic Acid)

According to an aspect of the present invention, the embryogenesis-inducing nucleic acid of the present invention contains a first coding region and a second coding region, wherein the first coding region encodes the first domain and the second coding region encodes the second domain, and the nucleotide sequence of the first coding region and the nucleotide sequence of the second coding region are linked to each other in-frame.

The first coding region of the embryogenesis-inducing nucleic acid of the present invention may have any nucleotide sequence without restriction, but may preferably have a nucleotide sequence identical or similar to the following nucleotide sequence.

*The nucleotide sequence of the Arabidopsis thaliana transcription factor TCP13 gene (SEQ ID NO:2)

The second coding region of the embryogenesis-inducing nucleic acid of the present invention may have any nucleotide sequence without restriction, but may preferably have a nucleotide sequence identical or similar to the following nucleotide sequence.

*The nucleotide sequence of the SRDX gene, which is derived from a transcription suppression region of the Arabidopsis thaliana SUPERMAN gene with modifications (SEQ ID NO:4)

Nucleic acids with nucleotide sequences similar to the first coding region and the second coding region mentioned above can also preferably be used as the embryogenesis-inducing nucleic acids of the present invention, since they are likely to encode fusion proteins having similar effects, i.e., embryogenesis-inducing proteins of the present invention, when expressed in plant cells due to their structural similarity.

Specifically, in the present invention, the nucleotide sequence of the first coding region of the embryogenesis-inducing nucleic acid may preferably have a sequence homology (preferably, a sequence identity) of, e.g., 90% or more, or 95% or more, or 96% or more, or 97% or more, or 98% or more, or 99% or more, with the nucleotide sequence of SEQ ID NO:2.

Likewise, in the present invention, the nucleotide sequence of the second coding region of the embryogenesis-inducing nucleic acid may preferably have a sequence homology (preferably, a sequence identity) of, e.g., 90% or more, or 95% or more, or 96% or more, or 97% or more, or 98% or more, or 99% or more, with the nucleotide sequence of SEQ ID NO:4.

According to an embodiment of the present invention, the embryogenesis-inducing nucleic acid of the present invention may preferably contain a promoter region. In this embodiment, the nucleotide sequences of the first coding region and the second coding region may operably be linked to the nucleotide sequence of the promoter region.

The promoter region of the embryogenesis-inducing nucleic acid of the present invention may have any nucleotide sequence without restriction, but may preferably have a nucleotide sequence identical or similar to the nucleotide sequence of a promoter selected from the following.

*The promoter region of the Arabidopsis thaliana TT12 (transparent testa12) gene (SEQ ID NO:5)
*The promoter region of the Cauliflower Mosaic Virus (CaMV) 35S gene (SEQ ID NO:6).

In particular, the Arabidopsis TT12 gene promoter (SEQ ID NO:5) has the effect of causing a gene to express specifically in the endodermis (especially its first and second layers), the 2n cells of maternal origin of the ovule. Therefore, it may be highly desirable to use the Arabidopsis TT12 gene promoter as the promoter region in the embryogenesis-inducing nucleic acid of the present invention, since this will make it possible to artificially induce adventitious embryogenesis from ovule mother cells.

Nucleic acids with nucleotide sequences similar to the promoter regions mentioned above can also preferably be used as the embryogenesis-inducing nucleic acids of the present invention, since they are likely to encode fusion proteins having similar effects, i.e., embryogenesis-inducing proteins of the present invention, when expressed in plant cells due to their structural similarity.

Specifically, in the present invention, the nucleotide sequence of the promoter region of the embryogenesis-inducing nucleic acid may preferably have a sequence homology (preferably, a sequence identity) of, e.g., 90% or more, or 95% or more, or 96% or more, or 97% or more, or 98% or more, or 99% or more, with the nucleotide sequence of SEQ ID NO:5 or SEQ ID NO:6.

According to another embodiment of the present invention, the embryogenesis-inducing nucleic acid of the present invention is configured in such a manner that when introduced into the genome of a seed plant, the nucleotide sequences of the first coding region and the second coding region is operably linked to the nucleotide sequence of an endogenous promoter region of the seed plant. Various conventionally known techniques can be used for targeted introduction of such foreign genes. Examples of endogenous promoter regions in seed plants include, but are not limited to, genes such as δVPE, ESP1, and TTI.

According to an aspect of the present invention, the embryogenesis-inducing nucleic acid of the present invention is configured in such a manner that the nucleotide sequences of the first coding region and the second coding region having the function to induce embryogenesis are transcribed and translated, under the control of the promoter region, specifically in an ovule to produce the embryogenesis-inducing protein of the present invention.

According to an embodiment of the present invention, the embryogenesis-inducing nucleic acid of the present invention may preferably contain a terminator region. In this embodiment, the nucleotide sequences of the first coding region and the second coding region may operably be linked to the nucleotide sequence of the terminator region.

The terminator region of the embryogenesis-inducing nucleic acid of the present invention may have any nucleotide sequence without restriction, but may preferably have a nucleotide sequence identical or similar to the nucleotide sequence of a terminator selected from the following.

*The terminator region of the Arabidopsis thaliana heat shock protein (HSP)gene (SEQ ID NO:7).
*The terminator region of the Agrobacterium nopaline-synthesis enzyme gene (SEQ ID NO:8).

Nucleic acids with nucleotide sequences similar to the terminator regions mentioned above can also preferably be used as the embryogenesis-inducing nucleic acids of the present invention, since they are likely to encode fusion proteins having similar effects, i.e., embryogenesis-inducing proteins of the present invention, when expressed in plant cells due to their structural similarity.

Specifically, in the present invention, the nucleotide sequence of the terminator region of the embryogenesis-inducing nucleic acid may preferably have a sequence homology (preferably, a sequence identity) of, e.g., 90% or more, or 95% or more, or 96% or more, or 97% or more, or 98% or more, or 99% or more, with the nucleotide sequence of SEQ ID NO:7 or SEQ ID NO:8.

The nucleic acid of the present invention may further contain one or more other gene elements. Examples of such other gene elements include antibiotic resistance gene sequences, restriction enzyme sequences, and homologous recombination sequences.

The nucleic acid of the present invention contains the first coding region and the second coding region encoding the embryogenesis-inducing protein of the present invention, and may also contain, as optional elements, a promoter region, a terminator region, and/or other gene elements. When the nucleic acid of the present invention contains, in particular, any optional elements in addition to the first coding region and the second coding region, it is typical that the first coding region and the second coding region are linked to each other in-frame, the promoter region is operably linked to the first coding region and the second coding region at its 5′ side while the transcription suppression region and/or the terminator region are/is operably linked to the first coding region and the second coding region at its 3′ side. With this arrangement, when the nucleic acid of the present invention is introduced into a seed plant cell, preferably incorporated into its genome, these gene elements operate in functional association with each other, whereby the embryogenesis-inducing protein of the invention is expressed autonomously. That is, the nucleic acid of the present invention may preferably be configured as a chimeric gene cassette.

### (4. Vectors)

The nucleic acid of the present invention may usually be used in the form of a vector carrying the nucleic acid in order to introduce it into plant cells and incorporate it into the genome. Such vectors carrying the nucleic acid of the present invention also constitute an aspect of the present invention.

Such a vector (the vector of the present invention) may have any form, i.e., a linear or circular form. Particularly preferred is a circular form, such as a plasmid form. Specific examples of the vectors include plant virus vectors and Agrobacterium vectors.

A method using the plant virus vector comprises in-vitro transcription of cDNA in a plant virus genome into which a target gene is inserted; inoculation of the resulting RNA as a vector into a plant to be infected therewith; and expression of the target gene in the plant by the proliferative capacity and systemic transfer ability of the virus itself. Examples of plant virus vectors include cauliflower mosaic virus (CaMV) vectors, cucumber mosaic virus (CMV) vectors, tobacco mosaic virus (TMV) vectors, and potato X virus (PVX) vectors.

A method using the Agrobacterium vector (T-DNA vector) is based on a technique using a transfer DNA (T-DNA) sequence of a Ti plasmid of Agrobacterium. The T-DNA sequence has a right border sequence (RB sequence) and a left border sequence (LB sequence) at two ends and incorporates genes in the region between these sequences into plant genomes. A T-DNA binary system in a combination of two plasmids, that is, a binary plasmid and a helper plasmid, through modification of Ti plasmid has already been established, and extensively used in introduction of foreign genes by genetic transformation of plants.

The nucleic acid according to the present invention may preferably be carried on the vector such that the nucleotide sequence encoding the embryogenesis-inducing protein is incorporated into the plant genome and functionally expressed.

When the nucleic acid according to the present invention has a promoter region and a terminator region in addition to the nucleotide sequence encoding the embryogenesis-inducing protein and functions as a chimeric gene cassette that can be autonomously expressed in plant cells, the vector does not necessarily have regulatory elements, such as promoters and terminators, and needs to have only the elements necessary for incorporation into plant genomes (e.g., flanking sequences for homologous recombination, or LB sequence and RB sequence of T-DNA).

In contrast, when the nucleic acid according to the present invention has no promoter region and no terminator region and functions as a chimeric gene cassette that can be autonomously expressed in plant cells, the vector preferably has not only the elements necessary for the incorporation into plant genomes but also regulatory elements, such as promoters and terminators, so as to induce the expression of the embryogenesis-inducing protein contained in the nucleic acid of the present invention. Alternatively, when the vector of the present invention may be incorporated into plant genomes, the nucleotide sequence encoding the embryogenesis-inducing protein contained in the nucleic acid of the present invention is functionally linked and cooperated with regulatory elements, such as promoters and terminators, in plant genomes such that the embryogenesis-inducing protein is expressed.

The vector of the present invention may be used in combination with any other helper plasmid as needed. Examples of the other helper plasmid include helper plasmids having a vir region for a T-DNA vector.

The vector of the present invention can be readily produced by an appropriate combination of various gene recombination techniques well known to those skilled in the art.

### (5. Embryogenesis Inducing Method)

According to an aspect of the present invention, the embryogenesis-inducing method of the present invention includes expressing the embryogenesis-inducing protein of the present invention in a seed plant. According to an aspect of the present invention, in the embryogenesis-inducing method of the present invention, expression of the embryogenesis-inducing protein of the present invention is achieved by introducing the embryogenesis-inducing nucleic acid of the present invention into the seed plant and making it express. According to an aspect of the present invention, introduction of the embryogenesis-inducing nucleic acid of the present invention into the seed plant is achieved using the embryogenesis-inducing vector of the present invention.

The method according to the present invention comprises introducing to express the nucleic acid or the vector of the present invention described above into the genome of a seed plant. Two or more nucleic acids or vectors according to the present invention may be used in combination.

The target seed plants are, not limited to, usually angiosperms. Non-limiting examples of the angiosperms include plant species belonging to, for example, Solanaceae, Fabaceae, Brassicaceae, Gramineae, Asteraceae, Nelumbonaceae, Rosaceae, Cucurbitaceae, and Liliaceae. Specific examples include tobacco, Arabidopsis thaliana, alfalfa, barley, kidney bean, canola, cowpea, cotton, corn, clover, lotus, lentil, Lupinus, millet, oats, pea, peanut, rice, rye, sweet clover, sunflower, sweet pea, soybean, sorghum, triticale, jicama, velvet bean, broad bean, wheat, wisteria, nut plants, redtop, leek, snapdragon, celery, groundnut, asparagus, Scopolia japonica, Avena fatua, hedge bamboo, oilseed rape, bromegrass, bush violet, camellia, hemp, red pepper, chickpea, chenopodium, witloof, citrus, coffee tree, job's tears, cucumber, pumpkin, Bermuda grass, duckweed, datura, urimibae, digitalis, Japanese yarn, oil palm, oleander, fescue, strawberry, geranium, day-lily, para rubber tree, henbane, sweet potato, lettuce, Lens esculenta, lily, linseed, ryegrass, tomato, origanum, apple, mango, cassava, Medicago polymorpha, African linaria, sainfoin, geranium, Chinese fountain grass, petunia, garden pea, green bean, timothy, bluegrass, cherry tree, buttercup, radish, currant, castor oil plant, raspberry, sugar cane, salpiglossis, Senecio, Setaria, white mustard, eggplant, sorghum, Stenotaphrum secundatum, cacao, Trifolium, blue melilot, wheat, and grape. Among these plants are preferred Arabidopsis thaliana, rice, corn, wheat and barley, and fruits.

The nucleic acid or the vector of the present invention described above may be introduced into the genomes of seed plants by any method. For example, the vector of the present invention may be biologically transmitted to seed plants or introduced into tissues of seed plants by, for example, an agroinfiltration process, a PEG-calcium phosphate process, an electroporation process, a liposome process, a particle gun process, and a microinjection process, to be incorporated into the genomes of seed plants. Alternatively, the nucleic acid of the present invention may be directly incorporated into the genomes of plants by a known process, such as a CRISPR/Cas9 system, without use of the vector of the present invention. In an alternative process, the nucleic acid or the vector of the present invention is introduced into a tissue fragment of a seed plant to cultivate the tissue fragment into a plant that is a re-differentiated individual having a genome into which the vector of the present invention is introduced and constantly expressing the nucleic acid of the present invention.

### (6. Recombinant Seed Plant)

According to an aspect of the present invention, the recombinant seed plant of the present invention is a recombinant seed plant in which the embryogenesis-inducing nucleic acid of the present invention or the embryogenesis-inducing vector of the present invention is incorporated in its cells. According to another aspect of the present invention, the recombinant seed plant of the present invention is a recombinant seed plant produced by the embryogenesis-inducing method of the present invention.

The method of the present invention can yield a recombinant plant that contains the nucleic acid of the present invention, which is incorporated into its genome and has nucleotide sequences encoding the embryogenesis-inducing protein, so as to express the embryogenesis-inducing protein. The recombinant plant can express the embryogenesis-inducing protein and thereby exhibit the ability to produce embryos without fertilization.

The subject plants of the present invention include transgenic seed plants produced by the method according to the present invention described above, transgenic seed plants containing the nucleic acids or the vector of the present inventions in their genomes, transgenic seed plants capable of developing functional endosperms without fertilization, and progenies or fragments of those plants. The term “progeny of plant” refers to a progeny produced by sexual or asexual reproduction of the plant and includes a clone of the plant. For example, the progeny of the plant can be produced from proliferation materials (e.g., seeds, fruits, ears, tubers, root tubers, stumps, calluses, and protoplasts) of the plant or its progeny. In the present invention, the term “plant or progeny, or part thereof” indicates a seed (including germinated seed or immature seed), an organ or part thereof (including leaf, root, stem, flower, stamen, pistil, and fragment thereof), plant cultured cells, callus, and protoplast in the plant or its progeny plant.

### EXAMPLES

The present invention will now be described in more detail with reference to Examples. It should be noted that the present invention is not limited to the following Examples and can be implemented in any form without departing from the spirit of the present invention.

In primer base sequences described below where capital and small letters are mixed, the capital letters indicate regions complementary to the nucleotide sequence in the target genes for amplification whereas small letters indicate additional sequences.

### [Summary]

In Example 1, the coding region of the Arabidopsis thaliana transcription factor TCP13 gene was used as the first coding region, the coding region of the SRDX gene, which is derived from the transcription suppression region of the Arabidopsis thaliana SUPERMAN gene via modifications, was used as the second coding region, and these first and second codon regions were linked to each other in-frame. The promoter region of the cauliflower mosaic virus (CAMV) 35S gene and the terminator region of the Arabidopsis thaliana heat shock protein (HSP) gene were each operably linked to these coding regions on the upstream side and on the downstream side, respectively, in such a manner that the first coding region and the second coding region were expressed under the control of the promoter and the terminator, thereby constructing a plasmid for transformation (Construct A) carrying the chimeric gene 35S Pro:TCP13-SRDX_HSP ter (Chimeric gene A). This construct was introduced into an Arabidopsis thaliana plant to create a transformation plant, whose morphology was observed and whose ability to nurture fertilized embryos was evaluated, whereby Construct A's effect of inducing ectopic adventitious embryogenesis in Arabidopsis thaliana was analyzed.

In Example 2, the same procedure as that for constructing Chimeric gene A in Example 1 was followed except that the promoter region of the CAMV 35S gene was replaced with the promoter region of the Arabidopsis thaliana T12 gene, which has the effect of causing a gene to express specifically in the endodermis (especially its first and second layers), the 2n cells of maternal origin of the ovule, thereby constructing a plasmid for transformation (Construct B) carrying the chimeric gene TT12 Pro:TCP13-SRDX_HSP ter (Chimeric gene B). This construct was introduced into an Arabidopsis thaliana plant to create a transformation plant, whose morphology was observed and whose ability to nurture fertilized embryos was evaluated, whereby Construct A's effect of inducing ectopic adventitious embryogenesis in Arabidopsis thaliana was analyzed. This construct was introduced into an Arabidopsis thaliana plant to create a transformation plant, whose morphology was observed and whose ability to nurture fertilized embryos was evaluated, whereby Construct B's effect of inducing ectopic adventitious embryogenesis in Arabidopsis thaliana was analyzed.

### [Materials and Methods]

### *Plant Materials and Incubation

In all experiments, L. Heynh, accession Col-0 was used as Arabidopsis thaliana. Arabidopsis thaliana seeds were treated with a sterile solution for seeds (20% hypochlorite, 0.002% Triton) for 5 minutes by shaking, washed 5 times with sterile water, and sown on MS agar medium (0.5% sucrose, 0.8% agar) prepared in round petri dishes (2×9 cm, IWAKI, Japan). The sown seeds were treated for vernalization in the dark at 4° C. for 2 days and then incubated under constant light conditions at 22° C. After 3 weeks of incubation, the plants were transplanted into soil (a mixture of vermiculite and soil mix) and grown under conditions of 22° C., 16 hours light and 8 hours dark.

### *Extraction of Plant Genomes

Genome extraction from plants was performed as follows: cut plant leaves were placed in a 5-mL tube, and 200 μL of DNA extraction buffer (200 mM Tris-HCl, pH 8.0, 50 mM NaCl, 25 mM EDTA, pH 8.0, 0.5% SDS) was added. The mixture was ground well, combined with 100 μL of phenol-chloroform solution (1:1), stirred well, and centrifuged at 14500 rpm for 5 minutes at room temperature. The supernatant was transferred to a new tube, an equal volume of isopropanol was added, stirred well, and centrifuged at 14500 rpm for 15 minutes at room temperature. The supernatant was removed, 400 μL of 70% ethanol was added, and centrifuged at 14500 rpm for 5 minutes at room temperature. The supernatant was again removed completely, and the residue was air-dried for 30 minutes, and combined with 30 μL of TE buffer (10 mM Tris-HCl, pH 8.0, 1 mM Na₂EDTA, pH 8.0) to prepare a genomic DNA solution.

### *Microscopic Observation

Stereomicroscopy was carried out using a Stemi 305, Stemi 2000-C, and Axioskop2 Plus system (Carl Zeiss Inc., Germany), with Axio Vision Rel. 4.5 as the resolution software. Fluorescence stereomicroscopy was carried out using an OLYMMPUS SZX10 (OLYMPUS, Japan), with cellSens standard as the resolution software. Differential interference microscopy was carried out using a Zeiss AxioImager 2 (Carl Zeiss Inc., Germany). Confocal laser microscopy was carried out using LSM 800 (Zeiss, Japan), FV3000RS and FV1000-D (OLYMPUS, Japan).

### *Pi Staining

Pi staining of plantlets in confocal laser microscopy was carried out using propidium iodide (Fujifilm Wako Pure Chemicals) prepared at 20 μg/mL in sterile water (Pi solution). Each sample was placed on a preparatory sheet and sealed with 40 μL of Pi solution for observation.

*RNA Extraction Total RNA was extracted using RNeasy Plant Mini Kit (Qiagen Inc., Germany) and DNA-free RNA was obtained by treating with RNase-Free DNase Set (Qiagen Inc., Germany). Plant samples grown on MS solid medium were crushed in liquid nitrogen, combined with 450 μL of RLT buffer (1% mercaptoethanol), and mixed well. The sample solution was placed in a purple column and centrifuged at 14500 rpm for 2 minutes at room temperature. The supernatant of the solution that passed through the filter was collected, combined with 250 μL of 99.5% ethanol, mixed well, and applied to a pink column, and centrifuged at 1000 rpm for 15 seconds at room temperature. 350 μL of RW1 was added to the column, and the mixture was centrifuged again at 10,000 rpm for 15 seconds at room temperature. The solution that passed through the filter was discarded, and the residue was then combined with 40 μL of DNase I solution, and let stand for 15 minutes. 350 μL of RW1 was then added to the column, and the mixture was centrifuged at 10,000 rpm for 15 seconds at room temperature. The filtered solution was discarded, and the residue was then combined with 500 μL of RPE buffer, and centrifuged at 10,000 rpm for 15 seconds at room temperature, 500 μL of RPE buffer was added again, and the mixture was centrifuged at 1000 rpm for 2 minutes at room temperature. The column was transferred to another tube, and centrifuged at 14500 rpm for 1 minute to thereby dry the membrane. The column was transferred to a storage tube, 40 μL of RNase-Free-Water was added, and the solution was centrifuged at 1000 rpm for 1 minute at room temperature so as to make the solution drop into the tube. This was used as the RNA extraction solution and stored at −80° C.
*qRT-PCR

qRT-PCR was carried out using FastStart Essential DNA Green Master (Roche Diagnostics Inc., Germany). The qRT-PCR reaction solution used was prepared by mixing 0.5 μL of reverse transcription solution, 4.3 μL of sterile Milli-Q water, 5 μL of 2×SYBR Premix Ex Taq II, and 0.2 μL of each primer. Samples were analyzed on a LightCycler (registered trademark) 96 SW 1.1 (Roche Diagnostics Inc., Germany).

### [Example 1] Construction of Construct a for Transformation (35S Pro:TCP13-SRDX_HSP Ter), Transformation of Arabidopsis thaliana Plant Using the Same, and Evaluation of the Resulting Transformant Plant A

### (1-1) Construction of Construct A

(a) Construction of Plasmid pBIG2

The transforming vector pBIG-HYG (Becker, Nucleic Acid Research, (1990), 18(1):203) transferred from Michigan State University, USA, was cleaved with restriction enzymes HindIII and SstI, and the cleaved product was subjected to agarose gel electrophoretic separation to recover a pBIG-HYG DNA fragment not including GUS genes.

Similarly, the plasmid p35S-GFP (Clontech Laboratories Inc., USA) was cleaved with restriction enzymes HindIII and BamHI, and the cleaved fragments were subjected to agarose gel electrophoretic separation to recover a DNA fragment containing a CAMV 35S promoter (hereinafter also referred to as “CaMV 35S promoter” as appropriate).

DNAs having the base sequences of SEQ ID NOs: 9 and 10 described below were synthesized by a conventional process, were heated at 70° C. for ten minutes, and then were annealed by natural cooling to yield a double-stranded DNA. This DNA fragment has a sequence where the restriction enzyme site BamHI and the restriction enzyme sites SmaI and SalI are linked to the 5′ end and the 3′ end, respectively, of the omega sequence derived from a tobacco mosaic virus (TMV) that enhances the translation efficiency. The use of this sequence can increase the expression efficiency of the gene present at the 3′ side and introduce restriction enzyme sites essential for subsequent construction of plasmid.

(SEQ ID NO: 9)
5′-GATCCACAATTACCAACAACAACAAACAACAAACAACATTACAATT
ACAGATCCCGGGGGTACCGTCGACGAGCT-3′
(SEQ ID NO: 10)
5′-CGTCGACGGTACCCCCGGGATCTGTAATTGTAATGTTGTTTGTTGT
TTGTTGTTGTTGGTAATTGTG-3′

The DNA fragment containing the CaMV 35S promoter and the double-stranded DNA as synthesized above were inserted into the HindIII and SstI sites, respectively, of pBIG-HYG not including the GUS genes to produce a vector carrying the CaMV 35S promoter S for transformation of plants. This vector is referred to as “plasmid pBIG2”.

(b) Construction of plasmid P35SRDX

Two complementary DNA fragments having the base sequences of SEQ ID NOs: 11 and 12 as described below were produced where GGG and a stop codon were attached to the 5′ end and the 3′ end, restrictively, of the nucleotide sequence of SRDX, which was the transcriptional regulatory domain of the gene SUPERMAN in Arabidopsis thaliana.

(SEQ ID NO: 11)
5′-GGGCTCGATCTGGATCTAGAACTCCGTTTGGGTTTCGCTTAAG-3′
(SEQ ID NO: 12)
5′-CTTAAGCGAAACCCAAACGGAGTTCTAGATCCAGATCGAGCCC-3′

These synthesized DNA fragments were annealed and inserted into the plasmid pBIG2, which was produced by cleavage with the restriction enzyme SmaI. The products were sequenced to screen a plasmid containing the SRDX introduced in the forward direction. This plasmid is referred to as “plasmid p35SRDX”.

### (c) Construction of Plasmid 35S Pro:TCP13-SRDX_HSP Ter (Construct A)

The cDNA of the transcription factor gene TCP13 in Arabidopsis thaliana as a template was subjected to the polymerase chain reaction (PCR) with a 5′ end upper primer having the nucleotide sequence of SEQ ID NO: 13 described below and a 3′ end lower primer having the nucleotide sequence of SEQ ID NO: 14 described below to amplify a partial sequence where the stop codon was removed from the full-length sequence of the gene TCP13. The PCR was repeated 30 cycles, each cycle including a denaturing reaction at 94° C. for one minute, an annealing reaction at 50° C. for one minute, and an extending reaction at 72° C. for three minutes.

(SEQ ID NO: 13)
5′-ATGAATATCGTCTCTTGGAAAGATG-3′
(SEQ ID NO: 14)
5′-CTACCATGTAACAACGAATATAATGTC-3′

The resultant amplification product without the stop codon of the gene TCP13 was cleaved with SmaI, recovered by agarose gel electrophoresis, and inserted into the plasmid p35SRDX described above by a conventional process. The resulting plasmids were sequenced by a conventional method to screen a specific plasmid that coincided with the gene TCP13 in a reading frame of SRDX from the plasmids containing the gene TCP13 introduced in the forward direction.

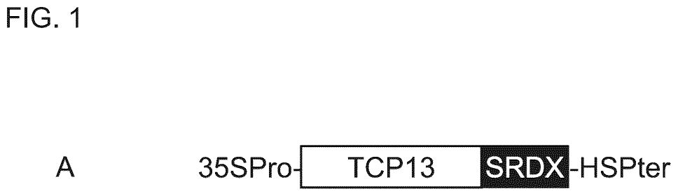

The resultant plasmid for transformation carries the chimeric gene 35S Pro:TCP13-SRDX_HSP ter where the promoter CaMV 35S, the gene TCP13, and the transcriptional regulatory domain SRDX are operably linked. This plasmid is abbreviated as “Construct A” as appropriate, and the chimeric gene 35S Pro:TCP13-SRDX_HSP ter carried on Construct A is abbreviated as “Chimeric gene A” as appropriate. The schematic structure of Chimeric gene A is shown in FIG. 1.

### (1-2) Development of Transformant Plant A

The Arabidopsis thaliana plants were transformed with Construct A by the method described in “Transformation of Arabidopsis thaliana by vacuum infiltration” (http://www.bch.msu.edu/pamgreen/protocol.htm). In this case, simple soaking without vacuum was used for transmission. Construct A was introduced into a soil bacterium (Agrobacterium tumefaciens) strain GV3101 (C58C1Rifr) pMP90 (Gmr) (koncz and Schell, Molecular and General Genetics, (1986), 204[3]:383-396) by electroporation. The bacteria were cultivated in 250 mL of LB medium for two days.

The bacterial cells were then collected from the culture solution and suspended in an infiltration medium (500 mL). Arabidopsis thaliana that had grown for 14 days was immersed in the solution for one minute, and then allowed to be regrown for seeding. The collected seeds were sterilized with 50% bleach and 0.02% Triton X-100 solution for seven minutes and then rinsed three times with sterile water, and the sterilized seeds were plated onto 1/2MS selective medium containing 30 mg/l hygromycin. Plants successfully transformed with Construct A acquired hygromycin resistance. The transgenic plants that grew in the hygromycin medium described above were screened and replanted to grow in soil. The plant transformed with Construct A is abbreviated as “Transformant plant A” as appropriate.

### (1-3) Phenotypic Observation of Transformant Plant a (Generation T1)

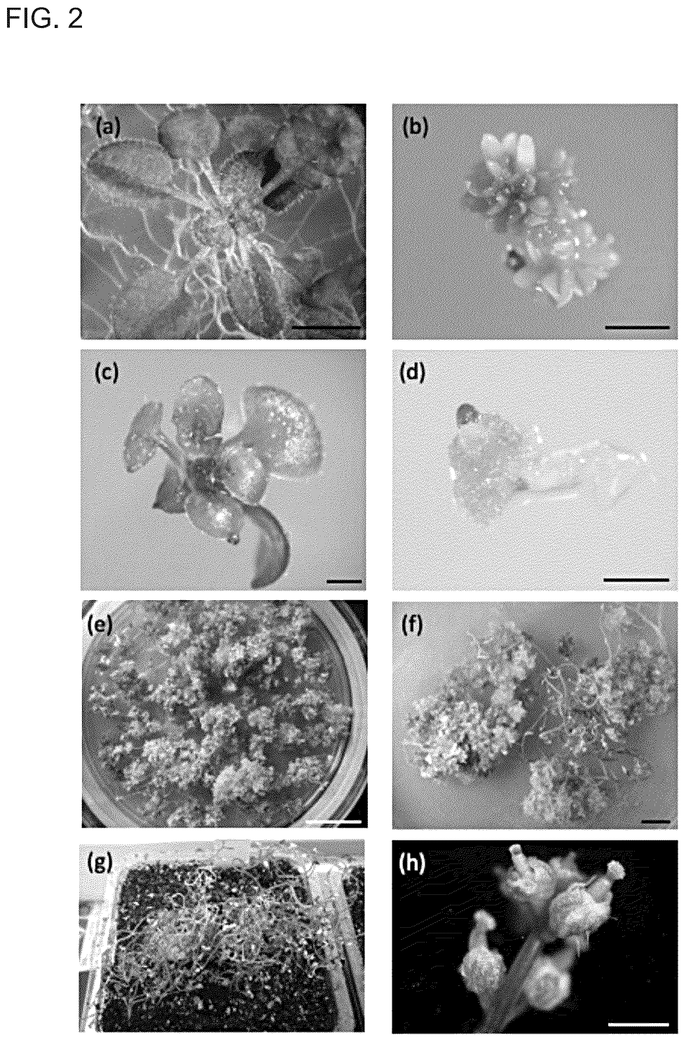

Twenty-five T1 generation transformed plantlets A were isolated and observed. As the formation of adventitious organs or callus was observed in all plants, the phenotype emergence rate was 100%. However, the shape of the adventitious organs formed varied and could be classified into several categories: those that looked like an aggregate of adventitious embryo-like structures, those that formed many abnormal true leaves, and those that looked like an aggregate of undifferentiated cells (callus) (FIGS. 2b to 2d). All Transformed plants A continued to proliferate for more than six months by repeated passaging on hormone-free MS agar medium (FIG. 2e). Not only simple cell proliferation but also various lines were observed during long-term proliferation, such as lines in which the same organs and tissues continue to form repeatedly and lines in which the differentiation state changes midway and produces completely different tissues. There was a case where calluses derived from a single line produced variations such as shoot-like structures, adventitious embryo-like structures, and other calluses (FIG. 2f). For example, when a line in which an adventitious embryo-like structure was initially observed was cultured successively, a portion of the tissue formed a true leaf and grew into a plant body. When this part was transplanted into soil and acclimatized (FIG. 2g), multiple flower stalks grew simultaneously and flowering was observed, but the morphology of the flower organs was defective, especially the pollen was low and sticky, and fertility was low (FIG. 2h). Thus, while the Transformant plants A had the common feature of continuing to propagate as calluses over a long period of time, the resulting calluses incidentally formed a variety of tissues, which differed among and within lines.

### (1-4) Gene Expression Analysis in Long-Term Cultured Callus of Transformed Plant a (Generation T1)

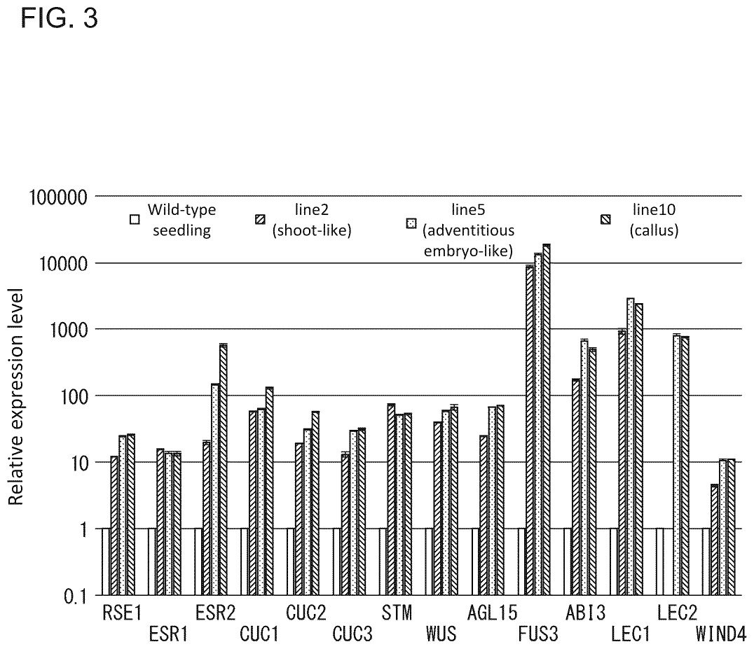

The expression of known adventitious organ-induced genes in a T1 generation Transformant plant A was analyzed using qRT-PCR. Specifically, the expression levels of 13 genes were analyzed: ESR1, ESR2, CUC1, CUC2, CUC3, STM, and WUS, which are involved in leaf and shoot differentiation; AGAMOS-like 15 (AGL15) FUSCA 3 (FUS3), ABA INSENSITIVE 3 (ABI3), LEC 1, and LEC2, which are embryogenesis-related factors: and WIND4, which induces callus formation upon injury. As a result, the expression level of each of these genes was markedly elevated compared to the wild type (FIG. 3). Thus, the long-term cultured callus of Transformed plant A can be said to be in a state of differentiation omnipotence, regardless of the observed morphology.

### [Example 2] Construction of Construct B for Transformation (TT12 Pro:TCP13-SRDX_HSP Ter) and Transformation of Arabidopsis thaliana Plant Using the Same

### (2-1) Construction of Construct B

The same procedure as that in “(1-4) Construction of Construct A” of Example 1 was carried out except that a DNA fragment containing the promoter region of the CAMV 35S gene was replaced with a DNA fragment containing the promoter region of the Arabidopsis thaliana TT12 gene, which was obtained by the following procedure, thereby constructing a plasmid for transformation (Construct B) carrying the chimeric gene TT12 Pro:TCP13-SRDX_HSP ter(Chimeric gene B).

Specifically, a cDNA containing the promoter region of the Arabidopsis thaliana TT12 gene was used as a template to amplify a partial sequence of the promoter region of the TT12 gene by PCR reaction, using a 5′ end upper primer with the nucleotide sequence of SEQ ID NO:15 and a 3′ end lower primer with the nucleotide sequence of SEQ ID NO:16. The PCR reaction was performed for 30 cycles, each cycle consisting of a denaturation reaction at 94° C. for 1 minute, an annealing reaction at 50° C. for 1 minute, and an elongation reaction at 72° C. for 3 minutes. The thus-obtained DNA fragment containing the promoter region of the TT12 gene of Arabidopsis was used instead of the DNA fragment containing the promoter region of the CAMV 35S gene in Example 1.

(SEQ ID NO: 15)
5′-ACTTGGCAAGATTATGTTCTGGTCAC-3′
(SEQ ID NO: 16)
5′-GGTCCGTTTATTAGTTCCTCTGTTTTTTTTTTCCCTTTTCT-3′

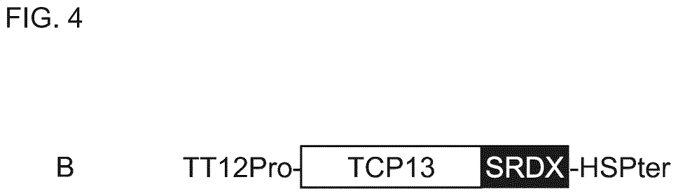

The thus-obtained plasmid for transformation carries the chimeric gene TT12 Pro:TCP13-SRDX_HSP ter, which includes the Arabidopsis thaliana TT12 promoter, the TCP13 gene, and the transcription suppression domain SRDX operably linked to each other. This plasmid is referred to as “Construct B” as appropriate, and the chimeric gene TT12 Pro:TCP13-SRDX_HSP ter carried by Construct A is referred to as “Chimeric gene B” as appropriate. The construction of the Chimeric gene B is schematically shown in FIG. 4.

### (2-2) Production of Transformant Plants B

The same procedure as that described in “(1-2) Construction of Transformed Plant A” of Example 1 was carried out except that Construct A was replaced with Construct B carrying Chimeric gene B mentioned above, whereby Arabidopsis plants transformed with Construct B were obtained. The resulting plants are referred to as “Transformant plants B.”

### (2-3) Production of Transformant Plant B (TT12)

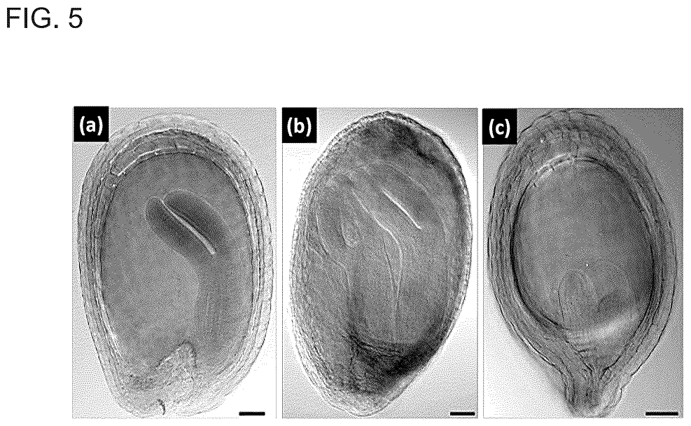

Seeds of a Transformed plant B were treated with chloral hydrate for transparency by the conventional method, and then observed using differential interference microscopy and confocal laser microscopy. No significant mutations were observed in seeds obtained from wild-type Arabidopsis (Col-0) (FIG. 5(a)) and those from the reference wild-type Arabidopsis (Col-0), as well as most seeds and fertilized embryos obtained from Transformed plant B, but dwarfing was observed in some plant bodies. In the observation of a developing seed, two torpedo-shaped embryos were observed. One of the two torpedo-shaped embryos was a normal torpedo-shaped embryo, while the other had a cell mass at the stem apex that appeared to be a true leaf or callus (FIG. 5(b)). The observation of another seed showed, in addition to a heart-shaped embryo that appeared to be fertilized an embryo, a spherical embryo-like structure (FIG. 5(c)).

### INDUSTRIAL APPLICABILITY

The present invention makes it possible to artificially and efficiently induce ectopic adventitious embryogenesis in seed plants without fertilization, and thus has high potential for use, especially in the field of agricultural production.

1. A method for inducing embryogenesis in a seed plant without fertilization, comprising expressing a protein having the function to induce embryogenesis in the seed plant,
wherein the protein having the function to induce embryogenesis is a fusion protein comprising a first domain and a second domain fused together,
wherein the first domain has an amino acid sequence with a sequence homology of 90% or more with the amino acid sequence of SEQ ID NO:1, and
wherein the second domain has an amino acid sequence with a sequence homology of 90% or more with the amino acid sequence of SEQ ID NO:3.

2. The method according to claim 1, wherein the expression of the protein having the function to induce embryogenesis is carried out by introducing a nucleic acid having a nucleotide sequence encoding the protein having the function to induce embryogenesis into the seed plant and expressing the protein having the function to induce embryogenesis in the seed plant,
wherein the nucleic acid comprises a first coding region and a second coding region, wherein the first coding region encodes the first domain and the second coding region encodes the second domain, and
wherein the nucleotide sequence of the first coding region and the nucleotide sequence of the second coding region are linked in-frame to each other.

3. The method according to claim 2, wherein the nucleic acid further comprises a promoter region, and wherein the nucleotide sequences of the first coding region and the second coding region are operably linked to the nucleotide sequence of the promoter region.

4. The method according to claim 2, wherein the nucleic acid is introduced into the genome of the seed plant in such a manner that the nucleotide sequences of the first coding region and the second coding region are operably linked to a sequence of an endogenous promoter region of the seed plant.

5. The method according to claim 3, wherein the protein having the function to induce embryogenesis is configured to be specifically expressed in an ovule under the control of the promoter region.

6. The method according to claim 2, wherein the nucleic acid further comprises a terminator region, and wherein the nucleotide sequences of the first coding region and the second coding region are operably linked to the nucleotide sequence of the terminator region.

7. A protein having the function to induce embryogenesis in a seed plant without fertilization, comprising a fusion protein of a first domain and a second domain fused together,
wherein the first domain has an amino acid sequence with a sequence homology of 90% or more with the amino acid sequence of SEQ ID NO:1, and
wherein the second domain has an amino acid sequence with a sequence homology of 90% or more with the amino acid sequence of SEQ ID NO:3.

8. A nucleic acid encoding the protein according to claim 7, comprising a first coding region, which encodes the first domain, and a second coding region, which encodes the second domain, wherein the nucleotide sequence of the first coding region and the nucleotide sequence of the second coding region are linked in-frame to each other.

9. The nucleic acid according to claim 8, further comprising a promoter region, wherein the nucleotide sequences of the first coding region and the second coding region are operably linked to the nucleotide sequence of the promoter region.

10. The nucleic acid according to claim 9, which is configured to be introduced into the genome of the seed plant in such a manner that the nucleotide sequences of the first coding region and the second coding region are operably linked to a sequence of an endogenous promoter region of the seed plant.

11. The nucleic acid according to claim 9, which is configured to specifically express the protein having the function to induce embryogenesis in an ovule under the control of the promoter region.

12. The nucleic acid according to claim 8, further comprising a terminator region, wherein the nucleotide sequences of the first coding region and the second coding region are operably linked to the nucleotide sequence of the terminator region.

13. A vector carrying the nucleic acid according to claim 8.

14. The vector according to claim 13, which is a plant virus vector or an Agrobacterium vector.

15. A recombinant seed plant capable of generating embryos without fertilization, comprising the nucleic acid according to claim 8 or a vector comprising the nucleic acid incorporated into its cells.

16. A recombinant seed plant produced by the method according to claim 1.
